(19) United States
(12) Patent Application Publication
(10) Pub. No.: US 2006/0079845 A1
(43) Pub. Date: Apr. 13, 2006

(54) Movable inflatable anchor for medical devices

(75) Inventors: Pamela A. Howard, Allentown, PA (US); Eben Howard, Allentown, PA (US); Steven C. Walker, Waxahachie, TX (US)

Correspondence Address: CAHN & SAMUELS LLP, 2000 P STREET NW, SUITE 200, WASHINGTON, DC, 20036, US

(73) Assignee: Eben Howard and Pamela A. Howard, Allentown, PA (US)

(21) Appl. No.: 11/149,433

(22) Filed: Jun. 10, 2005

Related U.S. Application Data
(63) Continuation-in-part of application No. 10/959,991, filed on Oct. 8, 2004.

(51) Int. Cl. A61M 5/32

(52) U.S. Cl. 604/175; 604/104; 604/101.05

(57) ABSTRACT

A medical device preferably includes a tube and at least one adjustable inflatable anchor. The anchor is capable of securing the tube to the patient to resist removal of the tube in the patient when the anchor is inflated within the patient. The anchor slidingly engages the tube such that the insertion depth can be selected by the user. In at least one embodiment, a second inflatable anchor is included for positioning outside of the patient.

This patent application is a continuation-in-part patent application of U.S. patent application Ser. No. 10/959,991, filed on Oct. 8, 2004, which is hereby incorporated by reference.

### I. FIELD OF THE INVENTION

The present invention relates to novel medical insertion devices and novel means for securing the devices during treatment of a patient. More particularly, the present invention relates to novel securable catheters and methods for securing same in and to a patient during treatment.

### II. BACKGROUND OF THE INVENTION

There are numerous situations in which it is desirable or necessary to secure a medical instrument in place in an incision, orifice or wound of a patient for drainage or to establish a passageway. This is true whether treating humans or other animals.

For example, as will be appreciated, a particular need for securing a medical device arises in the event of chest trauma. In the United States, chest injuries alone are responsible for one-fourth of all trauma deaths. On the battlefield, the death rate from chest wounds is even higher. Many chest trauma casualties could be prevented through early recognition of the injury followed by prompt management. In battlefield and mass casualty situations, the ability to quickly treat multiple patients is critical.

The lungs are surrounded by a pleural sac that consists of two membranes—the visceral pleurae and the parietal pleurae. The parietal pleura lines the thoracic wall, and the visceral pleura surrounds the lung. The pleural space is the space between these two layers of pleurae and contains a thin layer of serous pleural fluid that provides lubrication for the pleurae and allows the layers of pleurae to smoothly slide over each other during respiration events.

Pneumothorax is the medical condition resulting from air entering the pleural space. Hemothorax is the medical condition resulting from blood entering the pleural space. Both of these conditions can result from an injury or trauma to the chest. More importantly, pneumothorax and hemothorax are potentially lethal unless treated promptly. Common causes of pneumothorax and hemothorax include penetrating injuries (e.g., gunshot and stab wounds or injuries occurring as the result of a surgical procedure) and blunt injuries (e.g., from direct blows, crushing injuries, blasts, or falls). Pneumothorax may also occur as a result of the use of positive end-expiratory pressure (PEEP) in connection with mechanical ventilation procedures, or spontaneously as a result of emphysematous blebs (air spaces that may occur in the lung as a result of emphysema).

Normally, the pressure in the pleural space is much lower than the atmospheric pressure. Following trauma, air may enter the pleural space in several ways, e.g., through a communication between the pleural space and the outside air, or a leak from disrupted alveoli, bronchi or ruptured esophagus. The entry of air into the pleural space (pneumothorax) results in an increase in the pressure in the pleural space. The increase of pressure in the pleural space compresses the lung, which can cause a potentially fatal condition known as a collapsed lung.

Eliminating pneumothorax requires prompt decompression of the pleural space, usually accomplished by the insertion of a chest tube (or needle) and evacuation of the air. Similar procedures are followed during the occurrence of a hemothorax to remove blood from the pleural space. More specifically, in order to decompress the pleural cavity, a chest tube is inserted through the appropriate intercostal space, which is the area between adjacent ribs. Typically the intercostal space is approximately 1-2 cm in size. However, there are significant individual differences depending on the size of the individual, and the phase of the respiratory cycle (the intercostal spaces widen during normal inspiration). Furthermore, there are substantial regional size differences, e.g., the intercostal spaces are deeper anteriorly than posteriorly, and deeper between the superior than the inferior ribs. The lateral part of the intercostal space is the widest zone of the intercostal space (i.e., at the anterior axillary line). In addition to the differences in size from one individual to the next, the composition of the chest wall itself can vary from person to person and also differs based on the gender of the patient. The male chest wall is composed of a greater percentage of muscle tissue than the female chest wall. On the other hand, the female chest wall is composed of a greater percentage of adipose tissue than the male chest wall. Each intercostal space contains three muscles: the innermost intercostal muscles, the internal intercostal muscles, and the external intercostal muscles. In addition, each intercostal space contains a neurovascular bundle (intercostal vein, artery and nerve) that runs below the ribs. Further, the chest wall is covered superficially by muscles, connective tissue and skin. For example, the chest wall, in the fifth intercostal space, anterior axillary line is covered externally by the serratus anterior muscle. The chest wall thickness (CWT) is defined as the length from the thoracic epidermal surface to the parietal pleural lining of the lung. As with the intercostal spaces and chest wall composition, there can be a great variation in CWT from individual to individual and from location to location in the same individual. Further, the position of the patient can also affect the CWT; the CWT is a few millimeters less when the patient is in a reclined position (torso 45 degrees from horizontal) as compared with the same measurement taken when the patient is in the supine position.

The above-described physical differences between individuals must be considered when inserting a chest tube into a patient. There are several other key factors that come into play when inserting chest tubes, including insertion location, penetration angle, and depth. The primary goals of the tube insertion are to effectively evacuate the unwanted air/blood from the pleural space while also avoiding or minimizing injury to the intercostal neurovascular bundle, lungs and other internal structures. In addition, the chest tube must be well secured to the chest wall so that it cannot be accidentally dislodged, and it must also be easily removable once the pneumo/hemothorax is absorbed.

Several techniques are currently used to insert and secure a chest tube in place. Each of these prior art techniques typically involve relatively lengthy manual procedures that require sutures to secure the chest tube to the chest wall. The most common technique for inserting and securing chest tubes involves surgical preparation and draping at the site of the tube insertion (usually at the nipple level-fifth intercostal space, anterior to the midaxillary line on the affected side), administering of local anesthesia to the insertion site, and making a 2-3 cm horizontal incision. A clamp is inserted through the incision and spread until a tract large enough to accept a finger is created. Next, the parietal pleura is punctured with the tip of a clamp, and the physician places a gloved finger into the incision to clear adhesions and to confirm the presence of a free pleural space locally. The proximal end of the chest tube is clamped and the tube is advanced into the pleural space. As the chest tube is inserted, it is directed posteriorly and superiorly. In this position, the chest tube will effectively clear the pleural space of both air and blood. This process typically takes from 8-12 minutes depending upon the skill level of the medical professional and the situation and environment in which the medical professional is working.

Once the chest tube is appropriately in place (determined by listening to air movement using a stethoscope), the tube is preferably connected to a one-way valve, such as a Heimlich valve, in order to clear air/blood from the pleural space. The tube must then be sutured to the skin to prevent its movement from the desired location. Typically, the skin flaps are sutured together up to the sides of the chest tubes and the suture then is tacked around the chest tube. A dressing or gauze is then applied and the tube is taped to the chest. This process can take from 3-5 minutes (and even longer) depending upon the skill level of the medical professional, but this is a critical step in that over time the sutures will become loss from movement of or by the patient and the sutures will stretch in the wet environment both of which lead to an increase likelihood that the chest tube will become loose, which is why time is taken to secure the chest tube with sutures and a dressing.

Insertion and securing a chest tube using this standard technique can many times require more than 15 minutes to accomplish by a physician and requires extensive medical training to be performed properly. Further, while performing the procedure, the physician must attend to the patient receiving the chest tube and thus is precluded from attending to other patients, even in mass casualty situations. The need to suture the chest tube in place slows the process dramatically, because they need to be tight to counteract the eventual stretching and loosening along with the inherent aspect of the sutures becoming slippery from bodily fluid.

A cuffed endotracheal tube is not designed for this type of use, and in fact would not work effectively. First, the insertion end of the endotracheal tube is pointed such that it might inadvertently pierce an internal structure, for example, an organ (in the example above, the lung or the heart) or muscle tissue that might lead to over complications. Second, the balloon is fixed to the tube at a predetermined location such that most of the tubes have insertion depth marks so that the user can tell how far the insertion is, but the balloon itself can not be relocated along the tube to vary the insertion depth beyond the balloon. The lack of adjustment depth leads to inability to secure the endotracheal tube at an appropriate depth based on stethoscope listening.

Various other specialized techniques are known in the art for inserting a chest tube, including the use of a rigid trocar (a sharp-pointed instrument equipped with a cannula); “over-the-wire” techniques (involving the insertion of a needle, attached to a syringe, through an incision and into the pleural cavity, and the introduction of a guide wire used to guide the insertion of progressively larger dilators or angioplasty balloons, and finally a chest tube); peel-away introducers for the insertion of mini-thoracostomy tubes in patients with spontaneous pneumothorax; and disposable laparoscopic trocar-cannulae.

However, most of these techniques also require that an incision be made to initiate the insertion. As will be appreciated by one of ordinary skill in the art, an incision reduces the “snugness” of the device with respect to the chest wall. An incision therefore reduces the stability of the device which may cause the device to move, change the angle of penetration or result in an accidental disengagement of the device from the chest wall. Migration of a chest tube from its proper location is a particular problem experienced in the art. Even when the time is spent to suture a chest tube in place, the tube may migrate or be easily, albeit accidentally, pulled out. If the tube gets pulled out far enough another tube may need to be placed and the patient can experience complications such as a reaccumulation of air or fluid in the chest cavity. Also, if the tube is simply reinserted after migrating out, the patient may develop empyema from the bacteria resulting from an unsanitized tube being inserted into the incision.

Moreover, in some of these techniques, pointed instruments remain inserted in the patient during use. The sharp tips of these devices lie in the vicinity of internal organs, thereby increasing the possibility of injury resulting from these procedures or any migration of the device. These situations underscore the importance of having a reliable means of securing the instrument in the desired location and hindering migration. These drawbacks are exacerbated in situations where there are mass casualties, the procedures must be performed under field conditions, and/or where movement of the patient becomes necessary.

A variety of catheters (or tubes) are used for different medical purposes and each of the catheters are held in place by sutures and further reinforced by the use of dressings and adhesive tape. Medical purposes include providing drainage after surgery or to reduce swelling in a body part of the patient, checking for leaks, providing nutrition, and conduit for administrating medication and dyes for tests. Examples of catheters include feeding/drainage tubes (e.g., Gastrostomy tubes (G-tubes), Gastrojejunostomy tubes (G-J tubes), and Jejunostomy tubes (J-tubes)), drains such as the Jackson-Pratt drain and T-tube, intraventicular shunt, tracheotomy tube, intraabdominal tubes, peritoneal catheter, pigtail drain, diagnostic peritoneal lavage kit. Even in these uses, the chest tube is sutured into place and covered with a dressing that is further taped to the patient's skin.

Accordingly, there remains a long-felt, yet unresolved, need in the art for a quick and reliable method of securing medical devices. Likewise, there remains a need in the art for a quick and reliable means of securing and removing a medical device from a patient. There also remains a need for a non-suture means of securing medical devices in place and an improved method of preventing migration of medical devices.

### III. SUMMARY OF THE INVENTION

The present invention overcomes the serious practical problems described above and offers new advantages as well. One object of at least one embodiment of the invention is to provide a means for securing a medical instrument in an incision, orifice or wound of a patient. According to one object of at least one embodiment of the invention, one aspect of the invention is to provide a quick and reliable means of securing a medical device in place. According to one object, an aspect of at least one embodiment of the invention is to provide a means which also allow quick retraction of the device from the patient. According to any of these aspects of the invention, one advantageous feature of the invention is the provision of means for removably securing a medical device in place without the need for sutures.

These and other objects, aspects and features of the invention may be realized by the provision of an inflatable anchoring member. The inflatable anchoring member may have any suitable configuration adapted to allow the member to serve as an anchor to hold a device in place. In at least one embodiment, the anchor includes an annular member that may be inflated to constrict the circumference of a device passing therethrough. The annular member is preferably inserted inside an incision, orifice or the like, in a deflated state and then inflated to a size which prevents the anchor from exiting the area. The constriction of the anchor upon inflation hinders the device's migration.

According to an advantageous feature of at least one embodiment of the invention, the anchor member is configured to be moveable along a length of the device's body. In a preferred embodiment, the anchor is configured such that it can be positioned along the length of the body of the device, whereby upon inflation it holds the device at a desired depth. More preferably, the anchor is configured to also allow the device to be held in a desired orientation.

According to one advantageous feature of at least one embodiment of the invention, there is provided a device incorporating the anchor member(s) and means described above. According to another advantageous feature of at least one embodiment of the invention, there is provided a chest tube incorporating an inflatable anchor as described above.

According to at least one embodiment, the invention includes a medical device including: a catheter having an insertion end and a non-insertion end disposed on opposite ends, the insertion end defining at least one hole providing communication with an interior passageway through the catheter; an inflatable anchor in slidable engagement of an outer circumference of the catheter; and means for inflating the anchor; whereby inflating the anchor grips the catheter. According to at least one embodiment, the invention includes a method of securing the medical device in a desired position including: inserting the tube in a desired position within a patient chest; positioning the anchor parallel to an interior side of a chest wall of the patient; and inflating the anchor to a size suitable to restrict movement of the tube relative to the chest wall of the patient.

According to at least one embodiment, the invention includes a medical device comprising: a catheter having an insertion end and a non-insertion end disposed on opposite ends, the insertion end defining at least one hole providing communication with an interior passageway through the catheter; an inflatable anchor in slidable engagement of an outer circumference of the catheter; a fill line connected to the anchor; and a fill port connected to the fill line such that a passageway exists from the fill port through the fill line to the anchor. According to at least one embodiment, the invention includes a securing set comprising: the medical device, fill material is selected from a group consisting of saline, water and air, and means for delivering the fill material to the fill port of the medical device.

An object of at least one embodiment of the invention is to provide methods and means for using the anchors and devices of the present invention described above. A presently preferred method for using a tube embodiment of the invention includes the steps of moving an annular anchor member to a predetermined position on the body of the tube, inserting the tube to a predetermined depth such that the tube is inside the wall of the patient's skin, and filling the anchor with a fluid from a syringe from a fill line associated with the anchor. In another exemplary embodiment, a first anchor body is disposed inside the chest cavity and filled with a fluid and a second anchor body is positioned outside the chest wall and filled with a fluid.

Given the following enabling description of the drawings, the apparatus should become evident to a person of ordinary skill in the art.

### IV. BRIEF DESCRIPTION OF THE DRAWINGS

The present invention is described with reference to the accompanying drawings. In the drawings, like reference numbers indicate identical or functionally similar elements. The use of cross-hatching and shading within the drawings is not intended as limiting the type of materials that may be used to manufacture the invention.

FIG. 1 illustrates an exemplary embodiment of a tube device including an inflatable anchor according to the invention.

FIGS. 2A and 2B depict an exemplary embodiment of an inflated anchor member separate from a tube device.

FIG. 3A illustrates a side view of an exemplary embodiment of an uninflated anchor member on a tube device according to an invention.

FIG. 3B depicts a cross-section of the exemplary embodiment illustrated in FIG. 3A.

FIG. 3C illustrates a cross-section of an exemplary embodiment of a tube device according to the invention.

FIGS. 4A and 4B depict a side view of an exemplary embodiment of a portion of a tube device according to the invention.

FIG. 5 illustrates a side view of an exemplary embodiment of a portion of a tube device according to the invention.

FIG. 6A depicts a side view of an exemplary embodiment of a portion of a tube device according to the invention.

FIG. 6B illustrates a cross-section of the exemplary embodiment illustrated in FIG. 6A.

FIG. 7A depicts a side view of an exemplary embodiment of a portion of a tube device according to the invention.

FIG. 7B illustrates a cross-section of the exemplary embodiment illustrated in FIG. 7A.

FIGS. 8A-E depict different exemplary projection configurations for an anchor according to the invention.

FIG. 9 illustrates an exemplary embodiment of an anchor and a tube according to the invention.

FIG. 10 depicts an exemplary embodiment of a tube after deploying the anchor in the inside chest wall of a patient according to the invention.

FIG. 11 illustrates an exemplary embodiment of a tube after deploying a first anchor body inside the chest wall of a patient and a second anchor body outside the chest wall of the patient according to the invention.

### V. DETAILED DESCRIPTION OF THE DRAWINGS

The present invention is based, in part, on the discovery that alternatively inflatable and deflatable members associated with a device for insertion in an orifice could constrict the movement of the device until it is desired to adjust or remove the device. While the present invention will be described in connection with a tube 10 having an annular inflatable anchor 20, it will be readily apparent to one of ordinary skill in the art that the present invention can be applied to a multiplicity of fields and uses including insertion into a variety of cavities such as the chest, abdomen or intracranial region, for example, for providing delivery mechanism or reducing fluid buildup by draining an impacted area. While preferred, the fields should not be deemed limited to medical devices for veterinary or human treatment. Moreover, while preferred, the uses should not be deemed limited to tubes (or catheters).

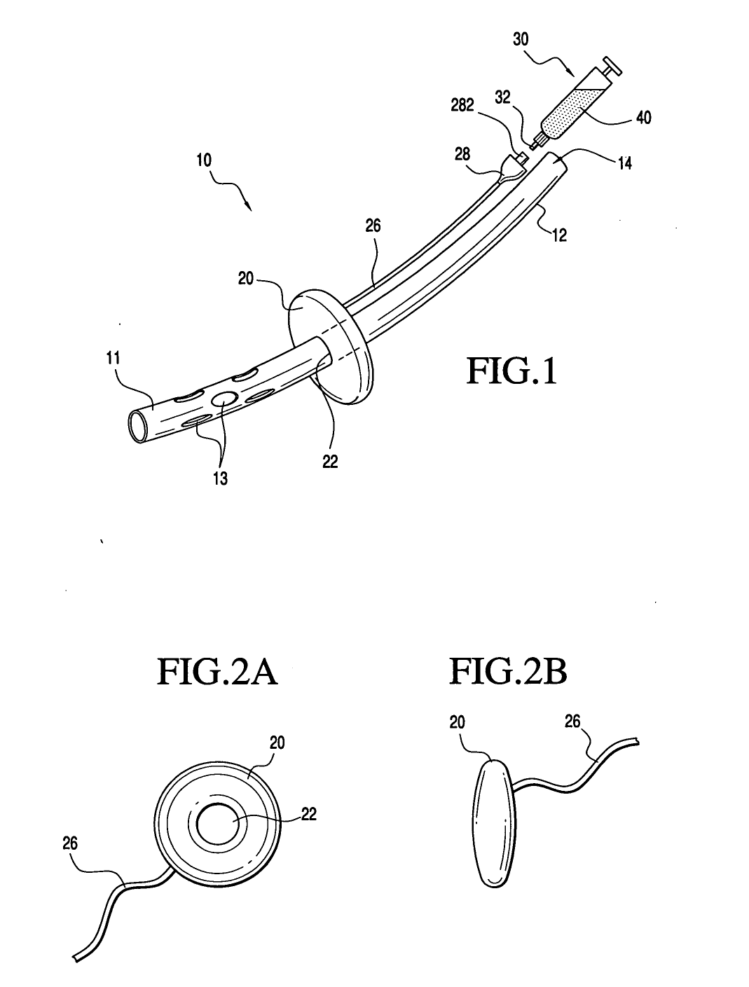

FIG. 1 illustrates an exemplary embodiment of the invention where tube 10 is a chest tube, although one of ordinary skill in the art will appreciate based on this disclosure that other types of tubes (or catheters) discussed above may be substituted for the illustrated chest tube. As shown, tube 10 includes insertion end 11 and non-insertion end 12. Insertion end 11 includes tube holes 13 providing communication with tube passageway 14 surrounded by wall 15. Tube passageway 14 in at least one exemplary embodiment is connected to a one-way valve (not shown) in order to assist clearing air/blood from the pleural space during pneumothorax or hemothorax treatment. The chest tube may include the one-way valve or the tube may simply be adapted for connection to a one-way valve or the like of a separate device.

Disposed on tube 10 is anchor member (or anchor) 20, which is moveable (such as slidable) along at least a portion of tube 10 including the entire length and alternatively be removable from tube 10 as illustrated in FIGS. 2A and 2B. The illustrated anchor member 20 includes an inflatable member having an annular configuration as illustrated, for example, in FIGS. 2A and 2B. Anchor member 20 is disposed around tube 10 such that tube 10 passes through the orifice 22 of anchor member 20.

The anchor member 20, for example, may be made with any suitable material capable of being filled with a fluid to expand its volume. Materials approved for medical use, such as those used in angioplasty balloons, are preferably used. Moreover, it is desirable for anchor member 20 to comprise a material and be configured to constrict the outer circumference of tube 10 in direct correlation to the increase in volume of anchor 20 due to the amount of fill material being supplied to the anchor's interior 24. The anchor member 20 although illustrated as an annular member may take a variety of other shapes while still providing the described functional capabilities.

Anchor member 20 is preferably moveable along at least a length of the outside of tube 10. As will be appreciated, by being moveable, anchor member 20 may be positioned inside the cavity of a patient after an appropriate length of the tube 10 is inserted. Thereafter, anchor 20 may be filled to secure the tube 10 in its desired location without the need for sutures.

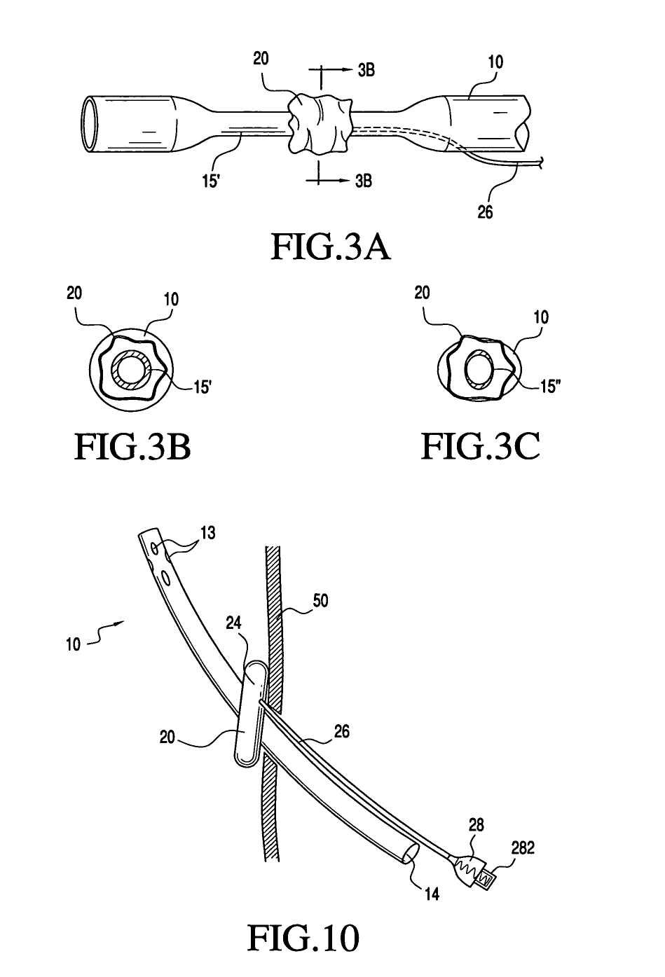

Tube 10 in at least one exemplary embodiment includes a section of a thinned or narrowed wall 15′ as illustrated in FIGS. 3A and 3B. This narrowed wall section 15′ provides an area for anchor member 20 to slide along and be restricted to a certain insertion depth. Thinned wall structure 15′ also is more easily deformed and gripped by an inflated anchor member 20. An alternative exemplary embodiment illustrated in FIG. 3C is to have the thinned wall section 15″ on either side of the tube such that a regular thickness wall continues along the entire length on opposing sides of the tube to provide additional rigidity for insertion of the tube into a patient. A further adaptation is to include a shape reinforcing member (not shown) to provide form and support to the tube such as a stiffener running along at least a portion of the length of tube 10.

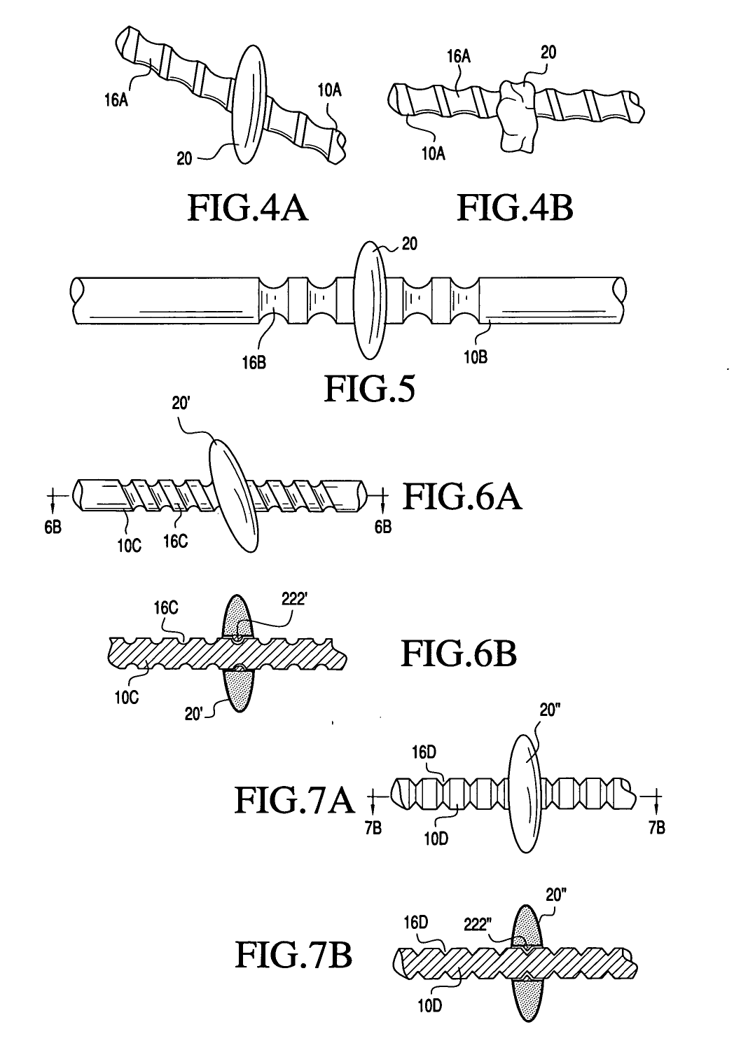

FIGS. 4A-7B illustrate additional exemplary embodiments of tube 10 (only a portion of tube 10 is illustrated) that include a variety of interfaces for engaging anchor 20. Although a limited number of interfaces are illustrated, one of ordinary skill in the art will appreciate based on this disclosure that a plurality of interfaces may be present in tube 10 over at least a portion of tube 10 most likely to be the area where anchor 20 would engage tube 10. FIGS. 4A and 4B illustrate a portion of tube 10A with a series of angled channels 16A (relative to the longitudinal axis of tube 10A) in wall 15 that an inflated anchor 20 fits into for securing tube 10A. The angle at which the channels are relative to passageway 14 is selected based upon the intended use of tube 10A. FIG. 4B illustrates a deflated anchor 20. The angled channels will allow tube 10 to pass through the patients dermal layers at an angle and be secured at that angle, which is beneficial particularly for chest tubes.

FIG. 5 illustrates tube 10B with channels 16B that are perpendicular to the passageway 14 of tube 10B. This arrangement of channels 16B is optimal where the intended use of the tube is substantially perpendicular to the patient's skin.

FIGS. 6A and 6B illustrate an exemplary embodiment where the surface of orifice 22′ includes protrusions or other projections 222′ for engaging channels 16C of tube 10C. Illustrated channels 16C are smaller than those illustrated in FIGS. 4A and 4B, because they are designed to engage protrusions 222′, which as illustrated are shaped to match the shape of channels 16C; however, the shapes of the protrusions may not match the shape of the channels. FIG. 6A also illustrates a slightly different angle than that illustrated for the exemplary embodiment shown in FIGS. 4A and 4B. FIGS. 7A and 7B illustrate triangular cross-section channels 16D and corresponding projections 222″.

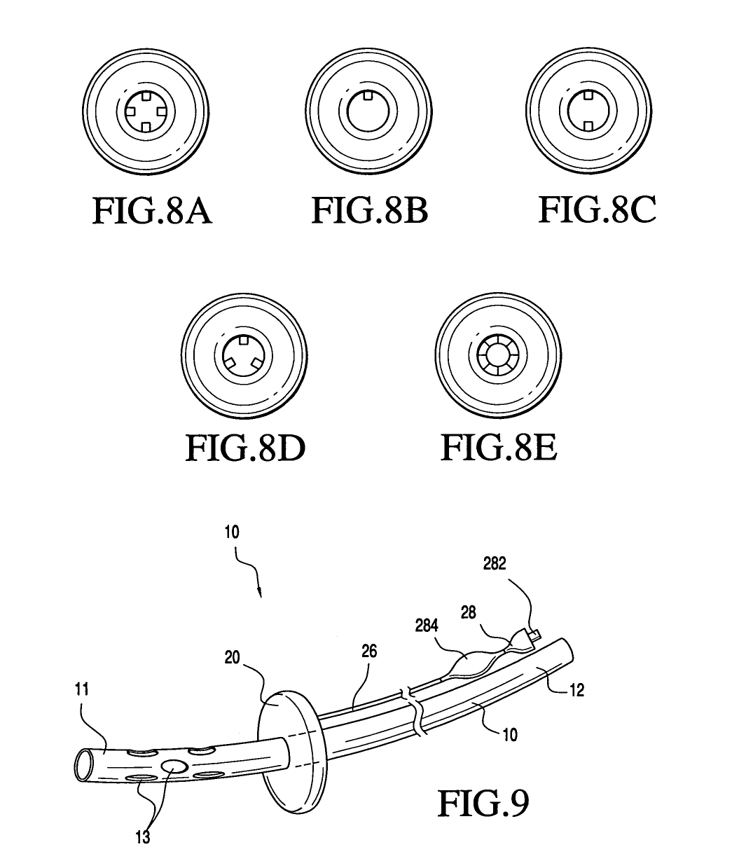

FIGS. 8A-E illustrate different exemplary configurations for protrusions 222. As illustrated, the number of protrusions can vary from at least one to nine, but may number more than nine. The protrusions may be spaced from each other as illustrated in FIGS. 8A-D or when anchor 20,is inflated form a ring as illustrated in FIG. 8E.

Fill material 40 for inflating the anchor 20 can be any suitable material. Preferably, the anchor member 20 is filled with a material non-toxic in the event of a rupture inside the patient's body. Presently preferred fill materials include fluids that can be delivered via a syringe (or means for delivering fluid) 30, such as water or air. Air is a presently preferred fill material due to the fact that it does not add appreciable weight to the anchor.

A fill line 26 preferably is associated with anchor member 20 for aiding the provision of fluid to the interior 24 of anchor member 20 as illustrated, for example, in FIGS. 1 and 9-11. Fill line 26 may be of any suitable construction which provides for fluid communication from a fill port 28 to anchor interior 22. Fill line 26 and fill port 28 together form a means for inflating anchor 20. Fill line 26 in at least one exemplary embodiment is formed (or contained) within the wall of tube 10 (not illustrated), either entirely, partially, or just a portion. Fill port 28 preferably includes a connection 282 for receiving the tip 32 of syringe 30 carrying fill material 40. More preferably, connection 282 is a spring valve 282 which allows communication to anchor 20 when actuated by tip 32 and which seals fluid-tight after retraction of tip 32 from fill port 28. Fill material 40 is deposited at fill port 28 until anchor member 20 is inflated to a volume suitable for holding a device, such as a tube 10, in a given orientation.

Fill port 28 may comprise a Luer-lock or like device for insuring a secure mating of the fill port and a syringe. FIG. 9 illustrates an exemplary embodiment where fill port 28 includes a pressure balloon 284 which serves as a proxy for determining the pressure of anchor 20 when anchor 20 is not visible to the user, e.g., when disposed inside a cavity of a patient.

Another exemplary embodiment includes at least one radiopaque marker at insertion end 11. Another exemplary embodiment includes a series of radiopaque markers spaced along at least a portion of tube 10. A further exemplary embodiment includes at least one radiopaque marker on anchor 20 to provide an indication of the inflation level of the anchor 20.

As depicted in FIG. 10, the anchor member 20 during use is positioned right inside the chest wall, intraabdominal wall or other inner cavity wall as applicable 50 and angled to be generally parallel to the wall 50.

In operation, the tube 10 is inserted using conventional steps for insertion up to suturing previously described herein. However, according to the present invention, the anchor 20 is positioned along the tube 10 in an area certain to be disposed inside the wall 50. Once the tube 10 is in its desired position (determined by listening to air movement using a stethoscope or alternatively using a radiopaque marker), the anchor 20 is positioned as described above by moving it along the tube 10 to a position adjacent wall 50 by pull along fill line 26. The anchor 20 may then be filled with the fill material 40 via the fill line 26 while the tube 10 is connected to a one-way valve or underwater-seal apparatus (not shown) in order to clear air/blood from the cavity such as the pleural space. When it is desired to remove or reposition the tube 10, the anchor 20 may be deflated by aspiration with a syringe inserted into the fill port 28.

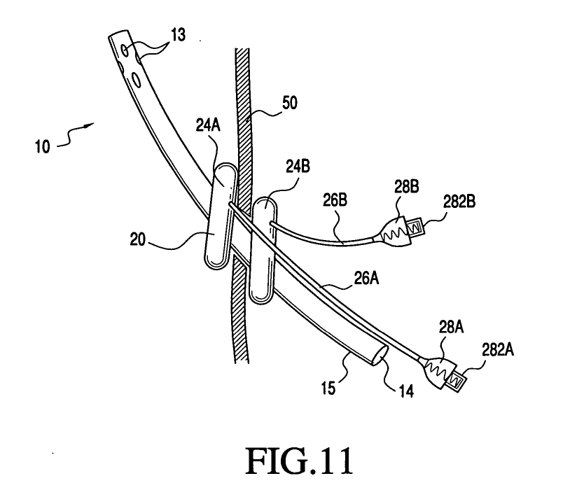

As shown in FIG. 11, an exterior anchor 20B may be used in combination (or to the exclusion of) the interior anchor 20A. According to this embodiment, each of the anchor bodies 20A, 20B have their own respective fill lines 26A, 26B and fill ports 28A, 28B. Such a construction allows for each anchor body to be positioned and inflated or deflated when desired. In addition, this construction allows for the anchor bodies to be used separately or together to anchor the tube depending on the circumstances. For example, it may be desirous to use an exterior anchor in situations where a patient must be moved, suturing is unavailable or impractical, or the fear of the anchor slipping deeper into, for example, the cavity are present. However, in an alternative embodiment, anchor bodies 20A, 20B share a common fill line. With that configuration, the exterior anchor's size and pressure is a fair indication of the interior anchor's size and pressure.

In operation, a preferred method of using the dual anchor body embodiment is the same as that for the single anchor embodiment with the exception of positioning and inflating the exterior anchor either before or after the chest tube is secured to the Heimlich valve or evacuation device.

As used above “substantially,” “generally,” and other words of degree are relative modifiers intended to indicate permissible variation from the characteristic so modified. It is not intended to be limited to the absolute value or characteristic which it modifies but rather possessing more of the physical or functional characteristic than its opposite, and preferably, approaching or approximating such a physical or functional characteristic.

The exemplary and alternative embodiments described above may be combined in a variety of ways with each other. Furthermore, the steps and number of the various steps discussed above may be adjusted from that discussed.

The present invention as described more fully above with reference to the accompanying drawings, in which preferred and exemplary embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. The accompanying drawings show exemplary embodiments of the invention.

Those skilled in the art will appreciate that various adaptations and modifications of the exemplary and alternative embodiments described above can be configured without departing from the scope and spirit of the invention. Therefore, it is to be understood that, within the scope of the appended claims, the invention may be practiced other than as specifically described herein.

We claim:

1. A medical device comprising:
a catheter having an insertion end and a non-insertion end disposed on opposite ends;
said insertion end defining at least one hole providing communication with an interior passageway through said catheter;
an inflatable anchor in slidable engagement of an outer circumference of said catheter; and
means for inflating said anchor; whereby inflating said anchor grips said catheter.

2. The medical device of claim 1, wherein said means for inflating comprises a fill line in communication with said inflatable member.

3. The medical device of claim 2, wherein said fill line is capable of receiving fill material from a syringe and depositing the fill material in an interior of said anchor, thereby inflating said anchor member and constricting movement of said catheter.

4. The medical device of claim 3, wherein said fill line includes a fill port comprising a spring-valve.

5. The medical device of claim 3, wherein the fill material is selected from a group consisting of saline, water and air.

6. The medical device of claim 5, further comprising a second inflatable anchor, said second anchor in slidable engagement of outer circumference of said catheter; and means for inflating said second inflatable anchor.

7. The medical device of claim 6, wherein said catheter includes a plurality of channels spaced from each other for engaging at least one of said anchors.

8. The medical device of claim 7, wherein at least one of said anchors includes at least one protrusion attached to the surface of said orifice for engaging at least one channel of said catheter.

9. The medical device of claim 7, wherein said channels are angled relative to the passageway.

10. The medical device of claim 1, wherein said catheter includes a section with a smaller outer circumference and walls thinner than the rest of said catheter.

11. The medical device of claim 1, wherein said catheter includes a plurality of channels spaced from each other for engaging said anchor.

12. The medical device of claim 11, wherein said anchor includes at least one protrusion attached to the surface of said orifice for engaging at least one channel of said catheter.

13. The medical device of claim 11, wherein said channels are angled relative to the passageway.

14. A method of securing the medical device of claim 1 in a desired position comprising:
inserting the tube in a desired position within a patient chest;
positioning the anchor parallel to an interior side of a chest wall of the patient; and
inflating the anchor to a size suitable to restrict movement of the tube relative to the chest wall of the patient.

15. A medical device comprising:
a catheter having an insertion end and a non-insertion end disposed on opposite ends;
said insertion end defining at least one hole providing communication with an interior passageway through said catheter;
an inflatable anchor in slidable engagement of an outer circumference of said catheter;
a fill line connected to said anchor; and
a fill port connected to said fill line such that a passageway exists from said fill port through said fill line to said anchor.

16. The medical device of claim 15, wherein said catheter includes a plurality of channels spaced from each other for engaging said anchor.

17. The medical device of claim 16, wherein said anchor includes at least one protrusion attached to the surface of said orifice for engaging at least one channel of said catheter.

18. The medical device of claim 16, wherein said channels are angled relative to the passageway.

19. The medical device of claim 15, wherein said fill port includes a spring valve.

20. A securing set comprising:
said medical device of claim 17,
fill material is selected from a group consisting of saline, water and air, and
means for delivering said fill material to said fill port of said medical device of claim 17.

21. A chest tube comprising:
an elongated tubular member having an insertion end and a non-insertion end disposed on opposite ends;
said insertion end defining a plurality of holes providing communication with an interior passageway through said tubular member;
said non-insertion end being adapted for facilitating the evacuating material from said insertion end through said passageway and out said non-insertion end;
an inflatable anchor disposed around an outer circumference of said tubular member;
means for inflating said anchor; whereby inflating said anchor grips said tubular member.
